(19) United States
(12) Patent Application Publication
(10) Pub. No.: US 2024/0023833 A1
(43) Pub. Date: Jan. 25, 2024

(54) SENSOR-BASED WALKING AID ADJUSTMENT SYSTEM

(71) Applicant: MOWA HEALTHCARE AG, Solothurn (CH)

(72) Inventor: Jan-Hagen SCHRÖDER, Solothurn (CH)

(21) Appl. No.: 18/027,157

(22) PCT Filed: Sep. 20, 2021
(86) PCT No.: PCT/IB2021/058547

§ 371 Date: Mar. 20, 2023

(30) Foreign Application Priority Data
Sep. 18, 2020 (DE) 10 2020 005 730.6

(51) Int. Cl. A61B 5/11; A61B 5/00

(52) U.S. Cl. CPC A61B 5/112; A61B 5/6828; A61B 2562/0219; A61B 2562/04

(57) ABSTRACT

The invention relates to a technical system for the analysis of motion data of a human being on the basis of one or more inertial measurement units (IMUs) with the possibility of data transmission to an evaluation computer. A first field of application can be the analysis for optimal medical patient supply with walking aids, such as ortheses and prostheses, with specific application specifications, which fully takes into account the patient's status, the medical diagnosis, and the orthopedic technical rules.

The invention relates to a technical system for analyzing human motion data. A first field of application can be the analysis for optimal medical patient supply with walking aids, such as ortheses and prostheses, with specific application specifications, which fully takes into account the patient's status, the medical diagnosis as well as the orthopedic technical rules.

### BACKGROUND OF THE INVENTION

Ortheses are medical aids for the support of functional limitations of extremities, for example as a result of cerebral palsy, foot drops, strokes, muscular dystrophies, or poliomyelitis. Ortheses allow the fixation of body parts for movement stabilization and/or protection as well as movement support of joints. Ortheses are applied externally to the extremity to be treated and worn for longer periods of time.

Prostheses, on the other hand, are medical devices used to replace amputated body parts of the upper and lower extremities and are therefore used on the body to compensate for the loss of function.

In order to be suitable for all possible movement restrictions, ortheses and prostheses must be individually adapted to each patient. Despite all the possibilities offered by modern technology, this requirement is not easy to implement, since the disciplines involved have very different approaches and their reliable coordination for the benefit of the patient often fails because of the costs involved. The result is often suboptimal care, which follows the cost pressure and often only succeeds or fails due to the random commitment of a person involved.

On the one hand, modular orthesis systems with a large number of adjustment devices for individual adaptation are currently developing into the state of the art. On the other hand, ortheses are still manufactured locally and highly individually after precise measurement of the patient's ergonomic conditions. However, despite all the progress in orthopedic technology systems and manufacturing processes, the correction result for many patients is still difficult to predict and is therefore still entirely dependent on the knowledge and experience of the orthopedic technician on site; there is no knowledge-based learning support system for the precise design of ortheses and prostheses based on locally collected patient data.

The probably most scientific method of mapping human movements is done by gait analysis [12]. In the gait lab, the spatial displacements of markers are recorded. With the resulting data, one is able to identify movement deficits, analyze them and assess the quality of support achieved. The physiologic gait pattern corresponds to a fluid locomotion and can be affected by disturbances of different origins [10). Such deviations can be quantified by gait analysis in order to design appropriate therapies. The gait analysis examines, among other things, movement patterns, acting forces as well as temporal and comparative parameters such as step cadence and symmetries [11].

VICON's gold standard is an optical tracking system consisting of several cameras that must be precisely installed and calibrated in space. The system enables highly precise measurements, but is not suitable for long-term monitoring of everyday activities due to the fixed installation. In addition, the system is expensive and takes up a relatively large amount of space. A smaller and less expensive method is gait analysis using walnut-sized inertial measurement units (IMUs). A single IMU can calculate its orientation in space. If one IMU per segment is fixed on the leg, the segments can be compared. This in turn allows the calculation of the movement of the legs and thus enables a flexible, location-independent gait analysis. These strong advantages are offset by the disadvantage of lower accuracy.

Particularly in the case of state-of-the-art orthesis systems, individual designs have to be defined before the orthesis is fabricated, which can lead to unnecessary restrictions in the fitting process, since alternative approaches for comparison are not economically feasible.

Only in EP 2 922 506 B1, however, is described a system with a very large freedom of application, which could serve for universal care.

In addition to the availability of widely modular systems with clearly defined and described physical properties, which is the prerequisite for building a database (DB), the necessary motion data must be collected just as clearly and physically unambiguously in all spatial dimensions. Measurement sensors are to be considered as state of the art: However, when used on humans, correct data acquisition can no longer be taken for granted. Today's common sensor systems are based on the integration of gravity as a means of orientation in space, which greatly impairs the agility of data collection due to the constant occupation of a measurement channel. In addition to the agility of the measurement systems, their complicated applicability is another obstacle to their widespread use in this medical field. Sensor systems that are characterized by spatial independence and maximum error tolerance in application are therefore desirable.

### OBJECT OF THE INVENTION

It is therefore the object of the invention to sensibly resolve the pointed-out desiderata in a closed system, namely the agile, gravity-independent data acquisition of human motion data by means of simple sensors and easy-to-use data acquisition stations and their fully comprehensive analysis according to medical and orthopedic-technical assessment parameters for reliable assessment and deficit care.

### BRIEF DESCRIPTION OF THE DRAWINGS

The invention is further exemplified by the following figures.

FIG. 1 shows the complexity of the human gait pattern with the many degrees of freedom in movement. The first row shows different phases of movement when walking in a side view. The second row shows the weight distribution on the soles of the feet during the movement phases shown in the first row. The third row shows the position of the pelvis during the movement phases shown in the first row (viewed from above). The fourth row shows the position of the pelvis during the movement phases shown in the first row (viewed from the front).

FIG. 2 shows the result of measurements with the system according to the invention: In the middle row are shown sections of the gait cycle. Thereabove are displayed measurements of the knee angle (right leg). Below are shown the measurements of the ankle angle.

FIG. 3 shows the measuring station with the screen of the first computer and various sensors on a stand (shown on the left). The sensors are manually attached to the subject's leg (right in FIG. 3).

FIG. 4 shows the positioning of sensors on a pro-band. In this example, 2 sensors on the feet, 2 sensors below the knee, 2 sensors in the middle of the thigh, and 3 sensors at the level of the pelvis (left, right (covered by the hand) and center) are used and evaluated for the gait analysis.

FIG. 5 shows measurement results of gait analysis of a patient with adjusted orthesis, i.e.,
a) measurement of the knee angle during the gait cycle, bottom left in FIG. 5,
b) measurement of the varus or valgus position of the knee joint during the gait cycle, top left in FIG. 5.

### DETAILED DESCRIPTION OF THE INVENTION

The solution of the object according to the invention consists essentially in providing a walking and adjustment system with a plurality of IMU sensors (IMU: inertial measurement unit), as detailed below.

Subject matter of the invention is thus a walking and adjustment system, comprising
1. one or more inertial measurement units (IMUs) with the possibility of data transmission to an evaluation computer,
2. an appropriate number of fastening devices to fix the inertial measurement units to different parts of a patient's body,
3. a measuring station with a first computer having a receiving device for the sensor data, which can simultaneously transmit the measured data to a second computer,
4. a second computer for the analysis of the measured data with a database to compare the measured data with data from other measurements,
5. a software for the analysis of the motion data, which runs on the second computer, where the analysis result is sent back to the first computer, and
6. a display on or connected to the first computer to display the analysis result.

The invention is based on the use of one or more of so-called inertial measuring units (IMUs), as they are known from other technical fields, for example from air navigation. Such IMUs have three orthogonal acceleration sensors (accelerometers) for the detection of translational movements in the three spatial axes and three orthogonal rotation rate sensors (gyroscopes) for the detection of rotational movements in the three spatial axes. For the purpose of the invention, micro-electromechanical systems can be used, which can be built in the form of integrated circuits.

Such IMUs are available on the market and need no further description here. Common models that can be used for the purpose of the invention are available, for example, from the following companies:
Gaitup (www.gaitup.com)
Cometa Systems (https://www.cometasystems.com/)
Axiamo GmbH (https://www.axiamo.com/)
Bosch Sensortec GmbH (https://www.bosch-sensortec.com)
Xsens (https://www.xsens.com/)
SBG Systems S.A.S. (https://www.sbg-systems.com/)

Decisive for the application according to the invention is the precision of the measurements of acceleration (accelerometers, A) and rates of rotation (gyroscopes, G). The measurements should, if possible, have at least the following measuring accuracies:
Sensitivity (A): ±2 g: 16384 LSB/g to (A): ±16 g: 2048 LSB/g
(programmable): (G): ±125 dps: 262.1 LSB/dps to (G): ±2000 dps: 16.4 LSB/dps
Digital resolution: (A): 16-bit or 0.06 mg/LSB
   (G): 16-bit or 0.004 dps/LSB
Sensitivity error: (A): ±0.4%
   (G): ±0.4% (with CRT)

Furthermore, the power consumption should be such that several hours of operation are possible with the help of common rechargeable batteries, so that long-term investigations are possible.

Preferably, the Axiamote X1 model of Axiamo is selected for the purpose according to the invention.

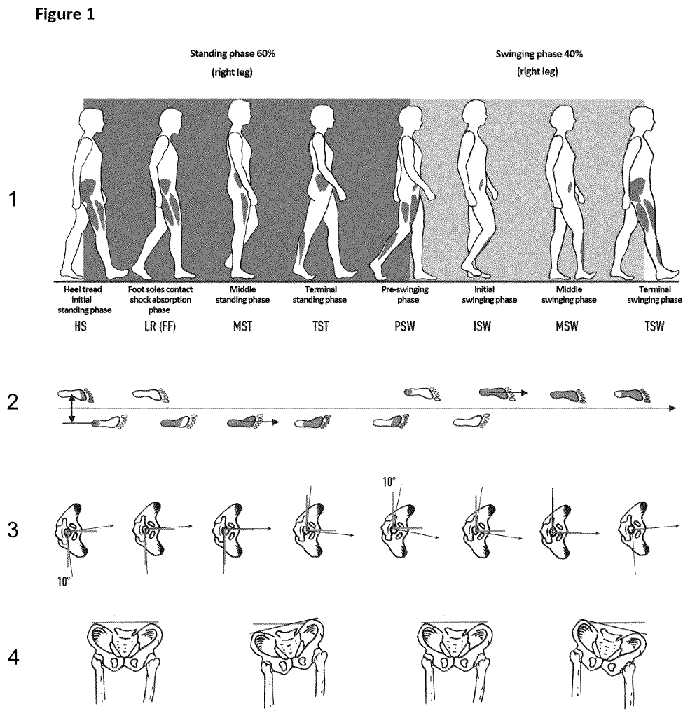

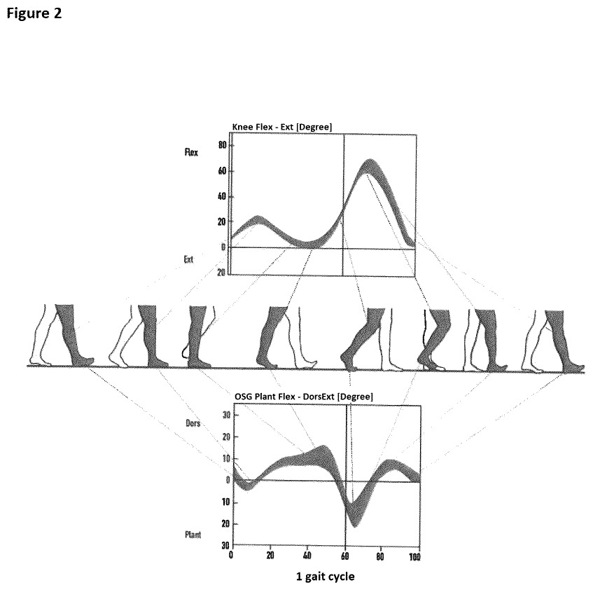

In particular, a single IMU sensor allows the complete description of the dynamic and static situation of a joint in relation to the physical parameters of orthopedics. The system uses a plurality of IMU sensors, the number of which is determined by the present medical diagnosis. The complexity of the human gait is shown in FIG. 1, with particular reference to the dynamic behavior of the pelvis, which in itself requires a number of sensors for medical and orthopedic analysis. The special, gravity-independent IMU sensors with their simple applicability are used here. The use of IMU sensors is also considerably more economical than today's standard sensors.

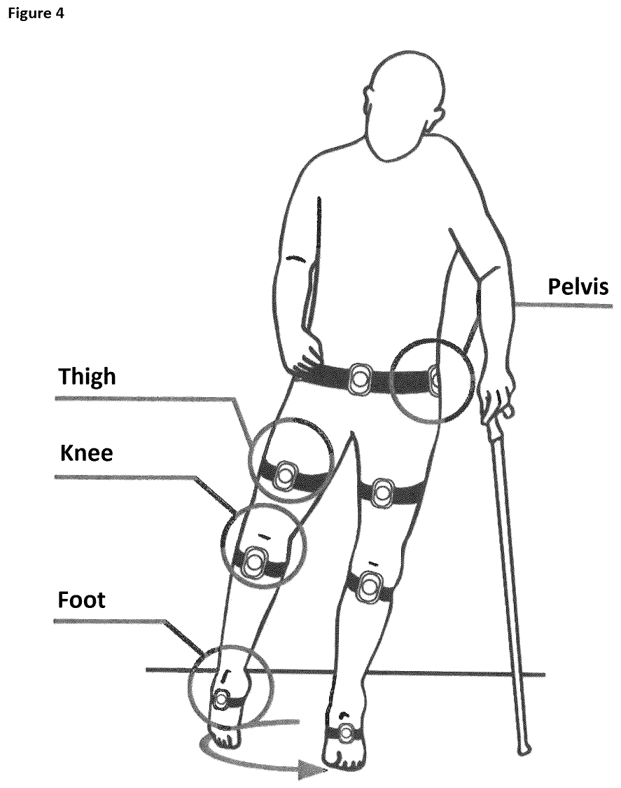

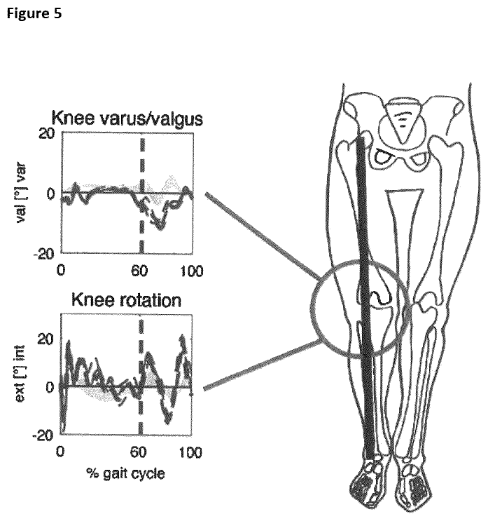

Due to their small size, the IMU sensors can be equipped with Velcro and/or magnetic fasteners so that they can be easily fixed to various body regions (FIG. 3).

According to the invention, the sensors are fixed to different parts of a subject's body. For gait analysis, for example, it has proven useful to use six sensors, two of which are placed on the lower leg, two on the upper leg, and two on the hip or iliac crest of the patient. Normally, they are fixed symmetrically on both halves of the body. In special cases, however, the arrangement can also be asymmetrical. FIG. 5 shows 9 sensors as an example. For certain simple analyses, a single sensor may be sufficient; in more complex cases, more than 9 sensors may be used. Of course, the amount of data increases with the number of sensors, but at the same time the quality of the measurements also increases.

When a patient moves, the sensors determine the accelerations occurring in the three spatial axes and the rotational movements. Using suitable analysis software (see below), the data of two sensors fixed to the same limb can be used to determine the temporal course of the joint angles.

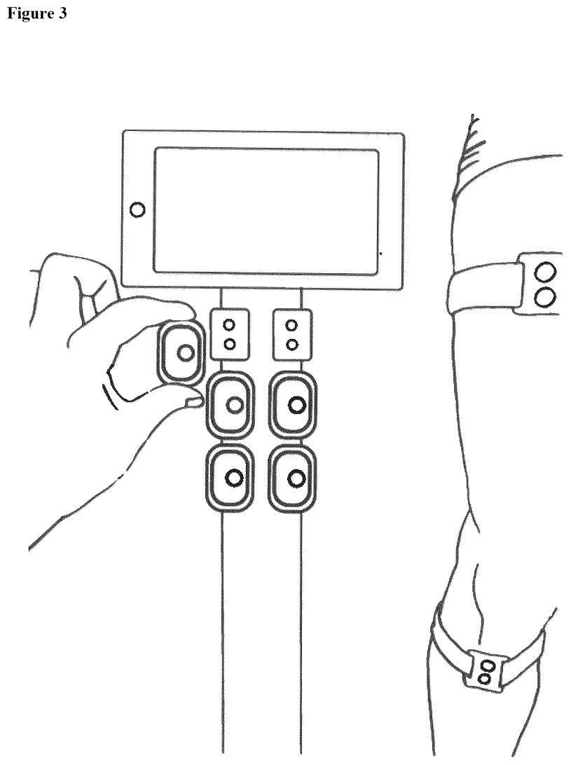

For example, a sensor on the thigh and a sensor on the lower leg can be used to determine the angle of the knee joint in the gait cycle in a time-resolved manner (see FIG. 2, top). By comparing the left leg with the right leg and comparing it with a patient who is not restricted in movement, deviations in gait behavior can be detected. The same applies to the analysis of the ankle joint angles and the angles in the hip joints.

Since the fastening devices are typically straps with Velcro closure, they can be easily applied to the bare skin as well as to garments and/or ortheses. This allows the gait pattern to be compared, for example, with and without orthesis.

Each sensor first transmits the respective data to the first computer for further evaluation. In principle, the transmission could of course be cable-based. For practical reasons, however, wireless transmission (via Near Field Communication (NFC), e.g., Bluetooth®) is preferable. A standard PC, a tablet PC, a smartphone, or similar can be used as the first computer.

Preferably, the actual analysis takes place in a larger data center, since the data analysis is computationally intensive. For this reason, the second computer is usually physically separated from the first computer. Analysis and storage of the data can also be realized virtually using cloud technologies. After the actual analysis has been carried out on the second computer, the motion data obtained is transmitted back to the first computer for display and further use. The display is usually visualized on the display of the first computer, if necessary with the aid of a connected monitor.

This division also allows the second computer to be designed in such a way that a large number of measuring stations are connected. This allows not only a better utilization of the second computer, but also the collection of as much data as possible in a database (DB) and the use of AI software, so that overall a self-learning system is created.

The measuring station for local data acquisition is the only locally required system hardware besides the IMU sensors, which means a comparatively low initial investment for the local end user. The measuring station is used for data acquisition and calibration prior to measurement (FIG. 3). Furthermore, the measuring station serves as a link between the IMU sensors and the corresponding analysis software on the second computer. The data transmission is made in a secured and anonymized manner (if necessary using local servers) to the data center for data analysis. In the course of data acquisition, further physiological data of the test persons can be recorded (e.g., height, weight, age, gender, girths at various points, etc.), which can be incorporated into the further analysis.

The software subsequently provides for the analysis process described below.

The actual motion data (e.g., the temporal course of the knee angle) is calculated from the acquired data by means of corresponding software on the second computer. The following methods are used for this purpose:

The program starts with the calibration of the sensors. Thereafter, the continuous loop is started, in which the data are measured, processed, and displayed. The individual steps are executed as follows.

### 0—Calibration

The gyroscope and the accelerometer in the IMU must be calibrated at startup to provide the most accurate measurements possible. This process usually takes a few seconds and can be performed using best practices, such as the Kalbr Library [1].

### 1—Measurement & Synchronization

The raw data of the individual IMUs (without magnetometer) are scanned by the controller and must then be synchronized for all IMUs used. The sampling rate should be as high as possible and can be downclocked later. For the synchronization, methods from Axiamo GmbH can be used. The synchronized data can then be sent to a more powerful computer, such as a tablet, for data processing.

### 2—Open-Loop Filtering

The raw data of the IMUs are pre-filtered by novel neural networks. This already allows a first reduction of the drift that can be expected from MEMS-based IMUs. Pre-filtering will also increase the precision of the conventional methods used in the next step.

Gyroscopes have already been successfully pre-filtered in drone applications [2] and this method is to be further advanced for human application.

Similar methods for accelerometers are not known to us and are to be researched within the framework of MOWA 4.0. Not only should the noise in the accelerometer signal be filtered, but also the acceleration due to gravity should be taken into account, so that the acceleration on the body minus the acceleration due to gravity can be measured.

### 3—Closed-Loop Filtering

The pre-filtered data are merged by sensor fusion using proven methods. The angular velocities of the gyroscope are transformed with linear accelerations of the accelerometer by methods like the Kalman filter [3] or complementary filter [4] to the orientation of the IMU sensor. With these methods, the XY plane can be determined correctly in the ideal case. For the determination of the remaining planes, however, the necessary information is missing (e.g., the measurement of the earth's magnetic field with magnetometer), whereby the Z axis of the IMU sensor is subject to a drift. With decision rules (heuristics), an axis common over all sensors can be determined under very good circumstances. A frequently used heuristic is the zero velocity update [5], which uses the step direction of the carrier as reference.

With the filtered data, the gait parameters can then be calculated.

### 4—Extracting the Gait Parameters

The gait parameters are extracted by proven methods [6, 7, 8]. For this purpose, the standstill of a leg is used as the beginning or end of a step. By the optional filtering of the accelerometer data by AI, a more precise calculation of the spatial parameters can be achieved, which can be calculated less precisely without filtering, due to the noise of the accelerometer.

### 5—Statistics & Representation

After calculating the gait parameters, the data can be displayed and interpreted, e.g., on an app or a tablet, similar to the system of Gait Up [9]. The statistics can then be saved and reused for further applications.

Each measurement can be stored in a database, so that over time a data collection is created, which can be further analyzed.

The value of this simplification cannot be overemphasized when considering today's gold standard of medical care, the gait lab:

In addition to the orthopedic technician, the physician and a very special high-speed camera technology including a computer center for image analysis is still required for the actual gait analysis. In addition, the gait lab must always complete a very complex gait learning phase at the start, in which the local reference gait must be established on site; the costs are correspondingly high and the use is locally limited.

Crucial for the medical usability is therefore the professional translation of medical classifications and diagnoses into usable algorithms.

All medically possible diagnoses, differential diagnoses, and contraindications are to be considered; orthopedically all classifications and techniques are to be considered. According to the invention, the systematic processing of the entire subject in its own algorithms is the key element for ubiquitous usability, which is qualitatively secured by the recourse to respective experts of the subdisciplines.

The data collected in this way, consisting on the one hand of the medical expertise and on the other hand of the measured gait data, provide a sufficiently good database for further data analysis with special algorithms. Subsequently, these data are converted into a 3D representation of the complete gait pattern.

This 3D representation allows, besides the simple and clear communication with the patient about his or her problems, the determination of the differential gait pattern to a local reference gait pattern. Due to the simple design of the system, the course of therapy can be measured and documented. Furthermore, the course of therapy can be compared to reference data of other subjects with the same symptoms.

Simplified, the goal of the 3D representation is the mathematical determination of the deviations from a reference set of data; these deviations are subsequently compensated for with the correct tools, for which the reference data of other subjects with the same symptoms are used.

The evaluation of the medical differential gait pattern must therefore lead to the definition of the medically correct result. On the basis of the calculated physical parameters, orthopedic-technical deficit compensation strategies can now be described.

In combination with the representations of the gait pattern, other medical aspects such as differential diagnoses and contraindications must be examined and lead to a system-based recommendation for orthopedic technical deficit compensation.

In other words, the system according to the invention does not only lead to a visual representation of the gait pattern on the basis of which aids (such as ortheses) are tested until a significant improvement of the gait pattern is achieved, as is known from prior art systems. Instead, the system according to the invention already suggests the most suitable aid itself (based on the measurements and the reference motion data). This eliminates the need for time-consuming trials to adapt aids with repeated gait analyses. The system according to the invention therefore avoids the repeated production of aids (e.g., ortheses), which do not or not sufficiently lead to an improvement of the gait pattern. The system according to the invention therefore further avoids the repeated gait analysis in a gait lab and avoids the related costs, the logistical effort and the frustration for the patients.

Ideally, after a simple application of the system in a measurement process, a standardized, and thus cost-effective, result is available.

According to the definition of orthopedic-technical deficit compensation, the knowledge of the technical possibilities represents the added value of the system according to the invention, since only in this way can the abstracted knowledge (and, if necessary, knowledge elaborated in own AI algorithms) become accessible to the patient's local provider. A database (DB) with components documented orthopedic-technically on a one-to-one basis thus rounds off the system, which again represents a decisive improvement in terms of cost and speed compared to the current gold standard gait lab. The gait lab can only give the medical recommendation, the solution must then still be worked out individually by the orthopedic technician. A database with direct access to the physical parameters of the care components is not yet available in this form.

Thus, the reduction of the error source “human” according to the invention has succeeded on all levels by consistent use of the system according to the invention. All knowledge-based monopolies in the process chain have been made ubiquitously available and objectified by the processing in DB. This has also leveled out the discipline-related perspectives in favor of higher-quality, faster and, last but not least, more cost-effective patient care.

Another advantage of the system is the significantly simplified and standardized documentation and communication based on the 3D representation.

For example, the simulation of the orthopedic technical deficit compensation using the 3D representation (if necessary according to AI recommendation) is extremely helpful for improving compliance and acceptance by the patient, since the desired gait pattern is immediately visually available.

Furthermore, the extrapolation of the data of the 3D representation in a future period allows a possible use in communication with the patient, which marks the contemporary use of CAx technologies in medicine.

A characteristic of the system according to the invention is the standardization of the processes, which enables the possibility of control history analysis over long periods of time. The heart of the system is the standardized measurement setup for constant data acquisition, which ensures data integrity.

A not to be underestimated advantage for the use in the practice is the documentation possibilities integrated in the system, which on the one hand can document the current treatment clearly and billable, but on the other hand also takes into account the temporal course of the therapy.

The accuracy of the measurements and the possibility of documentation also allow the system to be used outside of orthesis adjustment. For example, the course of neurological diseases, especially neurodegenerative diseases, can be tracked and documented. In this way, for example, the success of a drug therapy can be objectively recorded. In the context of such a treatment, systems according to the invention can be used, in which only 1 or 2 IMUs (e.g., at the wrist) are used. The measuring station with a first computer having a receiving device for the sensor data of the IMUs can be realized by a smartphone with a correspondingly adapted app, whereby the smartphone can transmit the measured data to a second computer for the analysis.

Furthermore, the AI algorithms that may be used can be described as self-learning, which in the long term will make the system increasingly “intelligent”. This also allows the possibility of transferring the principle to the prosthetic support, which would have to be realized by a separate prosthetic component DB.

In particular, it should be emphasized that—in contrast to prior art systems (e.g., DE 10 2007 052 806 A1)—no further mechanical sensors or aids are required to perform the desired walking aids adjustment. In particular, no mechanical scanning devices (calipers), force plates for recording ground reaction forces, no geomagnetic field sensors, and no cameras or other optical recording devices as described in DE 10 2007 052 806 A1 are required. For this reason, the system according to the invention can also be used outside a special measuring laboratory, e.g., during walks or runs on the streets or in nature.

In a preferred embodiment of the invention, the walking and adjustment system according to the invention comprises only the components listed below:
a) one or more inertial measurement units (IMUs) with the possibility of data transmission to an evaluation computer,
b) a number of fastening devices corresponding to the number of IMUs to fix the inertial measurement units (IMUs) to different parts of a patient's body,
c) a measuring station with a first computer having a receiving device for the sensor data of the IMUs, which can transmit the measured data to a second computer,
d) a second computer for the analysis of the measured data with a database and for optional comparison of the measured data to data from other measurements,
e) a software for the analysis of the motion data, which runs on the second computer, where the analysis result is sent back to the first computer, and
f) a display on or connected to the first computer to display the analysis result,
without the addition of further sensors and/or aids, such as scanning devices (calipers), force plates for recording ground reaction forces, geomagnetic field sensors, gravity sensors, cameras, or other optical recording devices.

In a further development of the system, the IMU sensors can collect further physiological data, e.g., measure vital functions of the test person (e.g., body temperature, pulse rate, blood pressure, oxygen saturation) of the test person or determine the exact location (e.g., via GPS data). This allows, for example, monitoring of the course of therapy, e.g., by measuring the distances walked. In this case, the first computer can be realized, for example, by a smartphone or tablet of the subject, and the forwarding of the data can be done via the Internet in real time.

### REFERENCES

[1] J. Rehder, J. Nikolic, T. Schneider, T. Hinzmann, and R. Siegwart, “Extending Kalibr: Calibrating the Extrinsics of Multiple IMUs and of Individual Axes,” in ICRA. IEEE, 2016, pp. 4304-4311. Accelerometer.
[2] Brossard, Martin & Bonnabel, Silvère & Barrau, Axel. Denoising IMU Gyroscopes with Deep Learning for Open-Loop Attitude Estimation. arXiv:2002.10718 [cs.RO]; 2020.
[3] Kalman, R. E. (March I, 1960). A New Approach to Linear Filtering and Prediction Problems. ASME. J. Basic Eng. March 1960; 82 (1):35-45.
[4] Andrien, A. R. P. & Antunes, Duarte & Molengraft, M. J. G. & Heemels, W. P. M. H. (Maurice). (2018). Similarity-Based Adaptive Complementary Filter for IMU Fusion. 3044-3049.
[5] Abdulrahim, Khairi & Moore, Terry & Hide, Christopher & Hill, Coralyn. (2014). Understanding the performance of zero velocity updates in MEMS-based pedestrian navigation. International Journal of Advancements in Technology. 5.
[6] Brégou Bourgeois, A., Mariani, B., Aminian, K., Zambelli, P. Y., & Newman, C. J. Spatio-temporal gait analysis in children with cerebral palsy using, foot-worn inertial sensors. Gait and Posture, 39(1); 2014.
[7] Kim, J. K., Bae, M. N., Lee, K. B., & Hong, S. G. Gait event detection algorithm based on smart insoles. ETRI Journal, 42(1), 46-53; 2019.
[8] Xing, H., Li, J., Hou, B., Zhang, Y., & Guo, M. Pedestrian Stride Length Estimation from IMU Measurements and ANN Based Algorithm. Journal of Sensors; 2017.
[9] Gait Up. PhysiGait. Gait analysis results generated by Gait Analyser 5.2. https://research.gaitup.com; Accessed Feb. 9, 2020.
[10] W. Pirker and R. Katzenschlager, Gait disorders in adults and the elderly: A clinical guide, Wiener Klinische Wochenschrift, vol. 129, no. 3-4, pp. 81_95, 2017.
[11] A. Muro-de-la Herran, B. García-Zapiraìn, and A. Méndez-Zorrilla, _Gait analysis methods: An overview of wearable and non-wearable systems, highlighting clinical applications, Sensors (Switzerland), vol. 14, no. 2, pp. 3362_3394, 2014.
[12] Perry J, Oster W, Wiedenhöfer B. (Hrsg.). Ganganalyse: Normen und Pathologie des Gehens. München: Urban & Fischer, 2003.

1. A walking and adjustment system, comprising
a) one or more inertial measurement units (IMUs) with the possibility of data transmission to an evaluation computer,
b) a number of fastening devices corresponding to the number of IMUs to fix the inertial measurement units (IMUs) to different parts of a patient's body,
c) a measuring station with a first computer having a receiving device for the sensor data of the IMUs, which can transmit the measured data to a second computer,
d) a second computer for the analysis of the measured data with a database and for optional comparison of the measured data to data from other measurements,
e) a software for the analysis of the motion data, which runs on the second computer, where the analysis result is sent back to the first computer, and
f) a display on or connected to the first computer to display the analysis result.

2. The system of claim 1, characterized by the use of 1 to 8, preferably 4 to 6 inertial measurement units (IMUs).

3. The system of claim 1, characterized by the use of Velcro and/or magnetic fasteners for separate fixation of the inertial measurement units (IMUs) on different body regions.

4. The system of claim 1, characterized by that each inertial measurement unit (IMU) transmits the collected measurement data wirelessly to the first computer.

5. The system of claim 1, characterized by that at least one inertial measurement unit (IMU) contains sensors for the collection of further vital data of the test persons.
